(19) United States
(12) Patent Application Publication
(10) Pub. No.: US 2018/0056758 A1
(43) Pub. Date: Mar. 1, 2018

(54) VEHICLE DUCT WITH ENHANCED LIGHTING OR CLEANING CAPABILITIES AND RELATED METHODS

(71) Applicant: FORD GLOBAL TECHNOLOGIES, LLC, Dearborn, MI (US)

(72) Inventors: Stuart C. Salter, White Lake, MI (US); Cornel Lewis Gardner, Romulus, MI (US); Paul Kenneth Dellock, Northville, MI (US); Annette Lynn Huebner, White Lake, MI (US)

(21) Appl. No.: 15/252,497

(22) Filed: Aug. 31, 2016

(51) Int. Cl. B60H 3/00; A61L 9/20

(52) U.S. Cl. CPC B60H 3/0078; F24F 2003/1667; A61L 9/205

(57) ABSTRACT

An apparatus for directing lighting and airflow to a vehicle cabin is provided. The apparatus comprises a light for generating ultraviolet light. A duct is provided for supplying airflow to the portion of the vehicle cabin and for receiving the ultraviolet light. At least a portion of the duct includes a functional modification adapted for interacting with the ultraviolet light for regulating an environmental condition of the portion of the vehicle cabin. The functional modification may provide luminescence, photocatalytic oxidation, or both.

### TECHNICAL FIELD

This document relates generally to the motor vehicle field and, more particularly, to a vehicle duct with enhanced lighting or cleaning capabilities and related methods.

### BACKGROUND

Vehicles are often provided with lights for lighting portions of the interior compartment. One location for such lights is in the portion of the vehicle cabin where passenger's feet are located when seated, such as the footwell in front seat or rear seat. Ducts are often provided at or near these locations as well to provide airflow for passenger comfort. While providing lighted ducts is known, it would be advantageous to provide an arrangement whereby the ducts may serve the dual purpose of providing enhanced lighting to the adjacent location, such as by providing luminescence, while also possibly cleaning the airflow provided.

### SUMMARY

According to one aspect of the disclosure, an apparatus for directing lighting and airflow to a vehicle cabin is provided. The apparatus comprises a light for generating ultraviolet light and a duct for supplying airflow to a portion of the vehicle cabin and for receiving the ultraviolet light. At least a portion of the duct includes a functional modification adapted for interacting with the ultraviolet light for regulating an environmental condition of the portion of the vehicle cabin, such as a footwell.

In one embodiment, the functional modification comprises a coating applied to an interior surface of the duct. The coating may be provided on at least one sidewall of the duct. The light may comprise an ultraviolet light emitting diode, which may be mounted on a printed circuit board connected to the duct. The portion of the duct may comprise titanium dioxide, a phosphor, or both. The duct may further include louvers positioned at an outlet of the duct, which louvers may include the functional modification.

According to a further aspect of the disclosure, an apparatus for directing lighting and airflow to a vehicle cabin is provided. A light for generating ultraviolet light is provided on or in duct for supplying the airflow to the vehicle cabin. The duct is at least partially adapted to luminesce upon receiving the ultraviolet light from the light.

In one embodiment, a portion of the duct comprises a luminescent coating applied to one or more sidewalls thereof. The duct may also be at least partially adapted for providing photocatalytic oxidation upon receiving the ultraviolet light. The disclosure also pertains to a method comprising activating the light to output light at a first level, and deactivating the light to cause the duct to output light at a second level lower than the first level.

According to a further aspect of the disclosure, an apparatus for directing lighting and airflow to a vehicle cabin is provided. The apparatus comprises a light for generating ultraviolet light. A duct is provided for supplying the airflow to the vehicle cabin and for receiving the ultraviolet light. The duct is at least partially adapted for providing photocatalytic oxidation upon receiving the ultraviolet light for cleaning the airflow provided to the vehicle cabin.

In one embodiment, at least a portion of the duct comprises titanium dioxide. At least a portion of the duct may be adapted for luminescing upon receiving the ultraviolet light. The light may comprise an ultraviolet light emitting diode positioned upstream of an outlet of the duct.

In the following description, several embodiments of a vehicle duct with enhanced lighting or air cleaning capabilities, as well as related methods, are shown and described. As it should be realized, the arrangement is capable of other, different embodiments and its several details are capable of modification in various, obvious aspects all without departing from the vehicle duct with lighting and cleaning capabilities, as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the disclosed vehicle duct with lighting and cleaning capabilities, serve to explain certain principles thereof. In the drawing figures:

FIG. 1 is a perspective view of a vehicle including a footwell having a lighted duct;

FIG. 2 is a schematic illustration of a duct according to one aspect of the disclosure;

FIG. 3 is a table illustrating the luminescence potential of various coatings; and

FIGS. 4-6 illustrate exemplary vehicle locations for functionally modified ducts according to the disclosure.

Reference will now be made in detail to the present preferred embodiments of a vehicle duct with enhance lighting or cleaning capabilities, examples of which are illustrated in the accompanying drawing figures.

### DETAILED DESCRIPTION

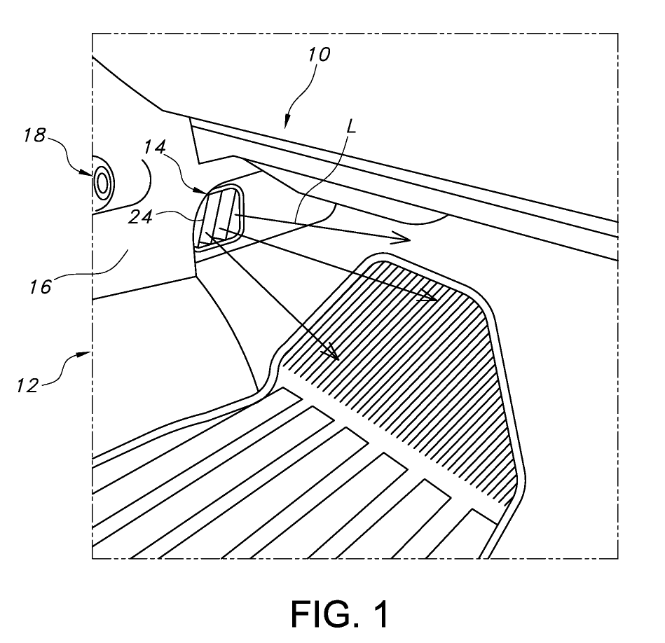

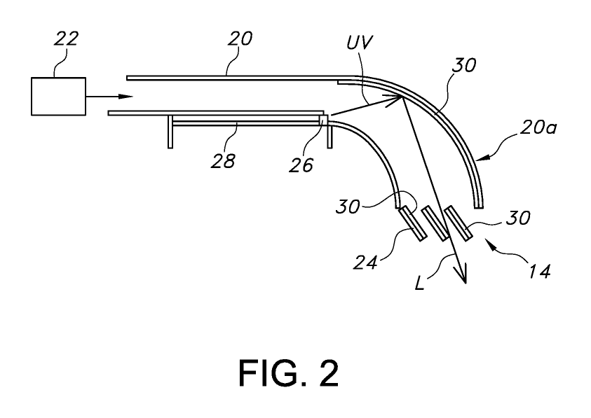

Reference is now made to FIG. 1, which illustrates a vehicle 10 including a cabin associated with a footwell 12 for receiving a passenger's feet when seated in a front seat (not shown in FIG. 1) of the vehicle. An outlet 14 is provided for providing airflow to the vehicle cabin, such as adjacent to a sidewall 16 forming part of a console 18. The outlet 14 may correspond to a duct 20 for supplying airflow from an air conditioning system 22 associated with the vehicle, as shown in FIG. 2. Flow regulators, such as louvers 24, may also be provided along the flowpath of the duct 20, such as at the outlet 14, for controlling the amount or direction of airflow provided to the cabin.

In addition to providing airflow, the duct 20 may also be associated with a light 26 for providing light through the outlet 14 to the vehicle cabin. The light 26 may comprise one or more light emitting diodes, or LEDs, arranged within a portion of the duct 20. In the embodiment of FIG. 2, the LED is part of a printed circuit board 28, or PCB, which circuit board may form a portion of the duct 20 for guiding the airflow from the system 22, and may be mounted in a releasable manner (such as by way of a snap-in connection). In this manner, the light 6 may be easily installed or replaced, and may also be used in a retrofit situation.

According to one aspect of the disclosure, the duct 20 may be provided with a functional modification for adapted for interacting with light output from the light for altering an environmental condition of the portion of the vehicle cabin. In one embodiment, the functional modification comprises adapting the duct 20 for luminescing in response to receiving light from the light 26. This may be achieved by providing a portion of the duct 20, such as a bend 20a adjacent to the outlet 14, with a coating 30 comprising a phosphor, and providing the light 26 with the ability to generate light in the ultraviolet spectrum, as indicated by arrow UV. The coating 30 may be applied to the duct 20, such as along all or part of bend 20a, as a paint (and may be provided as a top coating below another coating or layer, such as for example a bright white paint). Upon the ultraviolet light energy from the light 26 impinging the coating 30 including the phosphor, the energy is converted into visible light, as indicated by arrow L. This visible light L then emanates from the outlet 14 when at least partially opened for illuminating the vehicle cabin, such as along the footwell 12.

As illustrated, the arrangement may be such that the light 26 is arranged for directing the energy in the ultraviolet wavelength to the coating 30 at a first angle, and the resulting bend 20a in the duct 20 causing redirection of the visible light L to the outlet 14. As indicated, the flow regulators, such as louvers 24, positioned adjacent to or within the outlet 14 may also be provided with the coating 30 (additionally or alternatively), in which case the ultraviolet light UV reaching this location (such as by way of reflection) may also cause luminescence to occur. Such extraneous ultraviolet light UV exiting the outlet 14 may also be used to cause luminescence elsewhere in the vehicle cabin, such as on a part therein (e.g., part of a floor mat, carpet, trim, or the like, such as a logo or like indicia). In any case, the light output from the duct 20 may create a glow that provides a gentle light condition that enhances visibility without interfering with the visual perception of the passenger(s). The result is that the visible light L is provided to the cabin via the duct 20 to provide both enhanced lighting and airflow, if desired, without the need for a separate lighting source positioned elsewhere in the cabin apart from the duct (and the corresponding expense associated with a decorative lamp shell and lens).

According to a further aspect of the disclosure, the duct 20 may also be adapted for cleaning of the airflow provided to the cabin. This may be achieved by providing the duct 20 with a substance adapted for providing photocatalytic oxidation as a result of being contacted with light energy from the light 26. For example, the coating 30 may further comprise titanium dioxide (TiO₂), which may react with the ultraviolet energy from the light 26 to cause photocatalytic oxidation and thereby act as an air purifier.

This cleaning feature may be combined with the luminescent feature to advantage. For example, the coating 30 for providing photocatalytic oxidation may be combined with the luminescent material (phosphor) as noted above, such as to provide both the enhanced lighting and air cleaning functionality for regulating environmental conditions in the cabin. Titanium dioxide also provides a high refractive index, thus further helping to propagate the visible light L generated as the result of luminescence (if present) to the cabin. As indicated above, the coating 30 may be provided anywhere along the duct 20 within the output range of the ultraviolet light UV from the light 26, including on the louvers 24 (which may of course be used to regulate the light output from the duct, either manually or automatically, and also serve to conceal the light 26 from view). As also noted above, the coating 30 may be provided in layers, with one layer (e.g., a lower or inner layer) providing the photocatalytic oxidation and the other layer (e.g., the outer or upper layer) providing the luminescence.

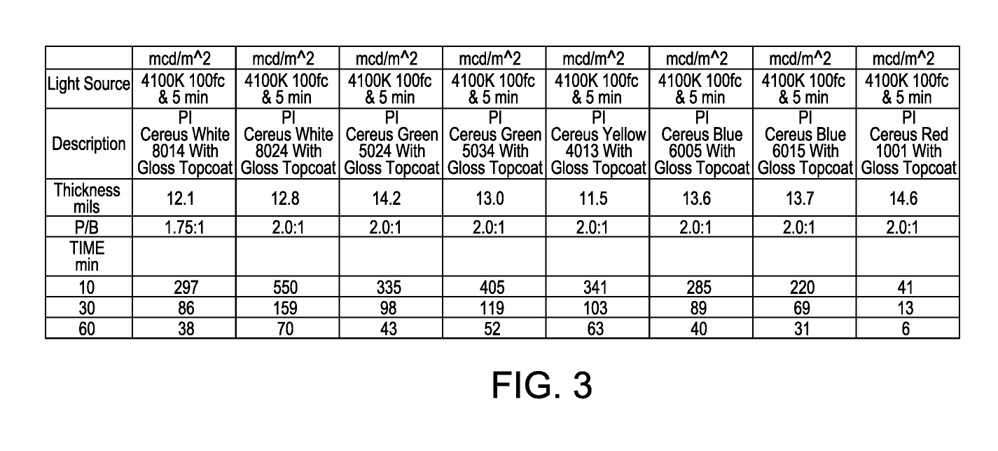

The amount and color of visible light V created by the luminescent duct 20 may be controlled based on the type of coating used, and may be regulated depending on whether the light 26 is powered or unpowered as part of a disclosed method of lighting a vehicle cabin. For instance, FIG. 3 is a table showing the use of different paints (including colors) as the coating 30, with different thicknesses and pigment-to-binder ratios and the results. The coatings referenced in these examples comprise CEREUS high intensity photoluminescent paints, available from Performance Indicator, LLC of Lowell, Mass. (MSDS PI-016 and PI-031 are incorporated herein by reference). As can be appreciated, the amount of candelas per square meter provided over time may thus be regulated accordingly (including for as long as one hour in the examples provided). Thus, the light L may be generated at a second, lower level for a relatively long time after the light 26 is turned off. This feature may be used to regulate the lighting condition of the vehicle 10, including after the vehicle power is turned off, thus providing a measure of accent lighting without consuming battery power.

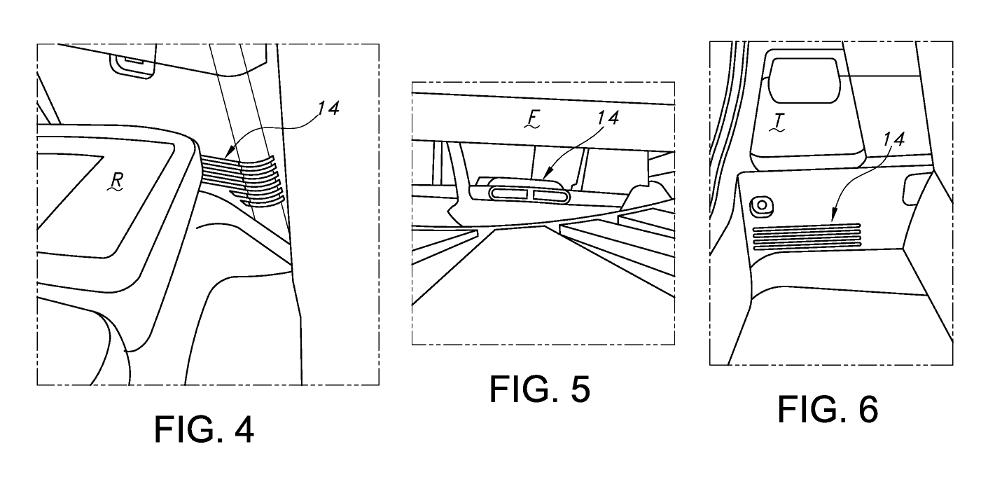

As can be appreciated, the lighted duct 20 according to this disclosure may be provided at other locations in the vehicle cabin. For example, FIG. 4 shows the duct 20 provided within the vehicle adjacent to a sidewall, such as along a rear seat R. FIG. 5 shows the duct 20 provided underneath a front seat F for directing the airflow and light either forward or rearward to a corresponding location. FIG. 6 shows that the duct 20 may be provided for illuminating a cargo location, such as along a liftgate T. Other variations are of course possible without departing from the scope of the disclosure.

In lieu of a coating 30, it is also possible to form a portion of the duct 20 with one or more materials for providing the desired luminescence or photocatalytic oxidation. For example, the duct 20 may be made of a polymer molded so as to include a luminescent material, such as a phosphor, and/or a photocatalyst, such as titanium dioxide.

In summary, a duct 20 may be functionally modified to provide lighting and airflow to a vehicle cabin. The duct 20 may be made luminescent by an applied coating 30 to provide for light through an outlet when the coating is bombarded by ultraviolet light UV from a source, such as a light 26 provided in the duct. The duct 20 may also be adapted to photocatalytic oxidation in the airflow therethrough and thereby act as an air purifier, which may be achieved by providing titanium dioxide in the coating 30. As a result, the duct 20 may serve the dual purpose of providing enhanced lighting to the adjacent location of the vehicle, such as a footwell, while also cleaning the airflow provided.

The foregoing has been presented for purposes of illustration and description. It is not intended to be exhaustive or to limit the embodiments to the precise form disclosed. Obvious modifications and variations are possible in light of the above teachings. For instance, while the coating 30 including both a phosphor and titanium dioxide according to the preferred embodiment is shown as being provided along a single bend 20a in the duct 20, it should be appreciated that the coating 30 may be separately provided at different locations, and may receive ultraviolet light from different sources or lights. In lieu of luminescence, a source of visible light may also be provided in addition to a UV light source for causing the photocatalytic oxidation. All such modifications and variations are within the scope of the appended claims when interpreted in accordance with the breadth to which they are fairly, legally and equitably entitled.

What is claimed:

1. An apparatus for directing lighting and airflow to a vehicle cabin, comprising:
a light for generating ultraviolet light; and
a duct for supplying airflow to the vehicle cabin, at least a portion of the duct including a functional modification for interacting with the ultraviolet light for regulating an environmental condition of the vehicle cabin.

2. The apparatus of claim 1, wherein the functional modification comprises a coating applied to an interior surface of the duct.

3. The apparatus of claim 2, wherein the coating is provided on at least one sidewall of the duct.

4. The apparatus of claim 1, wherein the light comprises an ultraviolet light emitting diode.

5. The apparatus of claim 1, wherein the light is mounted on a printed circuit board connected to the duct.

6. The apparatus of claim 1, wherein the portion of the duct comprises titanium dioxide.

7. The apparatus of claim 1, wherein the portion of the duct comprises a phosphor.

8. The apparatus of claim 1, wherein the portion of the duct comprises titanium dioxide and a phosphor.

9. The apparatus of claim 1, wherein the duct further includes louvers positioned at an outlet of the duct, the louvers including the functional modification.

10. A vehicle including the apparatus of claim 1.

11. The vehicle of claim 10, further including a footwell associated with an outlet of the duct.

12. A method of lighting the vehicle cabin with the apparatus of claim 1, comprising the step of luminescing a part of the vehicle cabin with the ultraviolet light emanating from the duct.

13. An apparatus for directing lighting and airflow to a vehicle cabin, comprising:
a light for generating ultraviolet light; and
a duct for supplying the airflow to the vehicle cabin and for receiving the ultraviolet light from the light, the duct being at least partially adapted to luminesce upon receiving the ultraviolet light from the light.

14. The apparatus of claim 12, wherein a portion of the duct comprises a luminescent coating applied to one or more sidewalls thereof.

15. The apparatus of claim 12, wherein the duct is at least partially adapted for providing photocatalytic oxidation upon receiving the ultraviolet light.

16. A method of lighting the vehicle cabin according to claim 12, comprising:
activating the light to cause the duct to output light at a first level; and
deactivating the light to cause the duct to output light at a second level lower than the first level.

17. An apparatus for directing lighting and airflow to a vehicle cabin, comprising:
a light for generating ultraviolet light; and
a duct for supplying the airflow to the vehicle cabin and for receiving the ultraviolet light, the duct being at least partially adapted for providing photocatalytic oxidation upon receiving the ultraviolet light for cleaning the airflow provided to the vehicle cabin.

18. The apparatus of claim 17, wherein at least a portion of the duct comprises titanium dioxide.

19. The apparatus of claim 17, wherein at least a portion of the duct is adapted for luminescing upon receiving the ultraviolet light.

20. The apparatus of claim 17, wherein the light comprises an ultraviolet light emitting diode positioned upstream of an outlet of the duct.
